(19) United States
(12) Patent Application Publication
(10) Pub. No.: US 2004/0063206 A1
(43) Pub. Date: Apr. 1, 2004

(54) Programmable scaffold and method for making and using the same

(76) Inventors: Jon A. Rowley, Chapel Hill, NC (US); Mohammad A. Heidaran, Cary, NC (US)

Correspondence Address: BECTON, DICKINSON AND COMPANY, 1 BECTON DRIVE, FRANKLIN LAKES, NJ, 07417-1880, US

(21) Appl. No.: 10/259,817

(22) Filed: Sep. 30, 2002

(51) Int. Cl. C12N 5/02; C12N 5/00

(52) U.S. Cl. 435/397; 435/395; 435/325

(57) ABSTRACT

A programmable scaffold which is a three-dimensional scaffold having interconnected pore structures and biologically active molecules physically entrapped therein. Preferably, the scaffold is a lyophilized hydrogel of crosslinked alginate or hyaluronic acid. The scaffold can be arrayed on a platform and loaded with various combinations of biologically active molecules for high throughput and high parallel screening and tissue engineering. A method for making and modifying the scaffold having steps of impregnating the scaffold with solutions of biologically active molecule and lyophilizing the impregnated scaffold.

### FIELD OF THE INVENTION

The present invention relates to scaffolds for cell culture and methods for making and using the same. Particularly, the present invention relates to three-dimensional scaffolds that are programmable with extracellular matrix (ECM) molecules and bioaffecting molecules for optimization of microenvironment for cell culture and tissue engineering.

### BACKGROUND OF THE INVENTION

Cell culture, as an important tool for biological research and industrial application, is typically performed by chemically treating the surface of cell culture device to support cell adhesion and bathing the adherent cells in culture medium containing supplements for cell growth. “Anchorage dependence” provides that the anchorage-dependent cells would only divide in culture when they are attached to a solid surface; the cells would not divide when they are in liquid suspension without any attachment. The site of cell adhesion enables the individual cell to spread out, capture more growth factors and nutrients, organize its cytoskeleton, and provides anchorage for the intracellular actin filament and extracellular matrix molecules. Thus, a surface that provides sufficient cell adhesion is vital to cell culture and growth.

In addition to cell adhesion and nutrients, hormones and protein growth factors are essential to support mammalian cell growth in cell culture. The requisite hormones and growth factors are contained in serum which is blood-derived fluid that remains after blood has clotted. Serum contains combinations of growth factors for cell growth. Mammalian cells deprived of serum stop growing and become arrested usually between mitosis and S phase, in a quiescent state called G₀. Various growth factors have been identified and isolated from the serum, however, it is still difficult to make the substitute for cell culture. Serum is expensive and needs to be replaced every 1-3 days, as the protein growth factors are quickly taken up by the fast growing cells. Thus, efforts have been made toward developing cell culture systems which promote cell adhesion and operate without the presence of serum.

Tissue engineering is a strategy for regenerating natural tissue. Cell culture in the context of tissue engineering further requires a three-dimensional scaffold for cell support. A scaffold having a three-dimensional porous structure is a prerequisite in many tissue culture applications, such as chondrocyte cell culture, because these cells would otherwise lose their cellular morphology and phenotypic expression in a two-dimensional monolayer cell culture. For regenerating natural tissue, the quality of the three-dimensional matrix can greatly affect cell adhesion and growth, and determine the success of tissue regeneration or synthesis. An optimal matrix material would promote cell binding, cell proliferation, expression of cell-specific phenotypes, and the activity of the cells.

Success in tissue engineering and transplantation of cells depends on the maintenance of the viability, differentiated phenotype, and integration with the body to deliver a desired therapeutic benefit. Maintenance and development of progenitor cells to functional tissue of every type requires different cell types, combination of cell types, physical environment, soluble environment, and proper cell signaling and cell interaction. High throughput and high parallel screening is required to find the suitable combination of microenvironment for tissue development.

A number of porous scaffolds for cell culture and tissue engineering have been disclosed in the literature. Shea et al. (Nature Biotechnology, Vol. 17, pages 551-554 (June 1999)) disclose highly porous three-dimensional poly(lactide-co-glycolide) scaffolds which are made by gas foaming and entrapped with plasmids. Petronis et al. (Journal of Materials Science: Materials in Medicine, 12, pages 523-528 (2001)) disclose a titania ceramic scaffold with topographic structure at sub-millimeter scale for hepatocyte in vitro culture; the titania ceramic is microporous, biocompatible, and inductive to cell aggregation, and the process for preparing the scaffold requires repeated oxidation, masking, and etching. Kim et al. (Fibers and Polymers 2001, Vol. 2, No. 2, pages 64-70) disclose a three-dimensional, porous, collagen/chitosan sponge made by lyophilization and crosslinking using EDC and NHS to increase biological stability and to enhance mechanical properties.

These scaffolds do not support cell adhesion. When strong cell adhesion is required, especially for those anchorage-dependent mammalian cell culture, the scaffolds must be modified to support cell adhesion. To solve the problem, cell adhesion-promoting molecules are immobilized onto the scaffold by covalent bonding so that cells can attach to these ligands. For examples, Kobayashi et al. (Biomaterials 1991, Vol. 12 October, 747-751) disclose covalent immobilization of cell-adhesive proteins onto surface of poly(vinyl alcohol) (PVA) hydrogel by diisocyanates, polyisocyanates, and cyanogen bromide to promote cell adhesion; Kobayashi et al. (Current Eye Research Vol. 10, No. 10, 1991, 899-908) disclose covalent immobilization of cell adhesive proteins and molecules on PVA hydrogel sheets to promote corneal cell adhesion and proliferation. Covalent modification adds complexity and steps to the process and may alter the desirable physical and chemical properties of the scaffold material and the ligands. It has been demonstrated that ECM molecules do randomly adsorb to hydrophobic polymers such as PGA, PLA, PCL, and all copolymers of polyesters, polyurethane, polystyrene. However, physical adsorption is difficult to control, which makes its use problematical in processes requiring constancy in promoting cell adhesion of a surface.

### SUMMARY OF THE INVENTION

The present invention provides a simplified method for making programmable scaffolds for cell culture with combinations of molecules promoting cell attachment or having cell signaling functions. The method involves the steps of impregnating a porous scaffold with a solution containing biologically active molecules, and lyophilizing the impregnated scaffold so that the biologically active molecules are entrapped within the porous scaffold. Preferably, the impregnated scaffold is washed to remove salts and pH adjusted, where necessary, prior to lyophilization.

The resultant porous scaffold permits three-dimensional cell or tissue culture and has an interconnected highly porous structure. The porous scaffold can be made from a variety of materials including polymers, ceramics, metal, or composites. These materials can be biocompatible, biodegradable or non-biodegradable. This attribute will depend on the ultimate use for the scaffold.

Acceptable polymers include alginate, hyaluronic acid, agarose, collagen, chitosan, chitin, polytrimethylene carbonate, poly hydroxybutyrate, amino acid-based polycarbonates, poly vinylchloride, polyHEMA, polystyrene, PTFE, poly ethylene glycol, or polypropylene glycol-based based polymers. Biodegradable polymers include poly lactides, glycolides, caprolactones, orthoesters, and copolymers thereof.

The porous scaffold is typically a lyophilized hydrogel of the polymer, including crosslinked alginate or hyaluronic acid.

The biologically active molecules include extracellular matrix (ECM) molecules, functional peptides, proteoglycans and glycoproteins capable of signaling cells, growth factors, molecules for optimal cell function, and combinations thereof. ECM molecules include fibronectin, laminin, collagen, thrombospondin 1, vitronectin, elastin, tenascin, aggrecan, agrin, bone sialoprotein, cartilage matrix protein, fibronogen, fibrin, fibulin, mucins, entactin, osteopontin, plasminogen, restrictin, serglycin, SPARC/osteonectin, versican, von Willebrand Factor, polysacchride heparin sulfate, cell adhesion molecules including cadherins, connexins, selectins, or combination thereof. Growth factors include epidermal growth factor, fibroblast growth factor, platelet-derived growth factor, nerve growth factor, transforming growth factor-β, hematopoietic growth factors, interleukins, and combination thereof. Typically, a combination of an ECM and growth factor(s) is selected for use. This permits the attachment of a specific cell type in close proximity to the growth factor, which permits the study of the interaction or controlled growth or selection. A microenvironment can be created. The programmable scaffold permits the study of events associated with the triggering of highly specific biological responses in cells through activation or inhibition of signal transduction pathways.

It is also possible with the programmable scaffolds to control and maintain the viability, phenotypic, and genetic expression of various cells for a variety of purposes including tissue engineering and also to use the programmable scaffolds in screening processes including high throughput and parallel screening methods.

The present invention further provides a method for making an array of scaffolds having the steps of distributing a solution of a suitable polymer on a platform to form solution spots, crosslinking the solution spots to form spots of crosslinked hydrogel, and lyophilizing the spots of crosslinked hydrogel to form an array of scaffolds. Preferably, the suitable polymer is hyaluronic acid or alginate. The crosslinking reaction mixture contains a diamine and a carbodiimide. The carbodiimide can be EDC at an amount of about 25% to 200% molar ratio of functional groups to hyaluronic acid or alginate, and preferably, about 50% to 100% molar ratio of functional groups to hyaluronic acid or alginate. The diamine, such as lysine or adipic dihydrazide, is at an amount of about 2% to 100% molar ratio of functional groups to hyaluronic acid or alginate, and preferably, about 10% to 40% molar ratio of functional groups to hyaluronic acid or alginate. The hydrogel solution may further comprise a coreactant which is HoBt, NHS, or sulfo NHS, at a ratio of about 1:50 to 50:1 to the carbodiimide, and preferably, about 1:10 to 4:1 to the carbodiimide (EDC).

The programmable scaffolds and arrays containing the same can be a component of a kit. The kit typically is designed to facilitate use and handling in the context of a desired operation, e.g. cell or tissue culture, screening operations. One or more of the other necessary reagents for the operation can be included along with written directions. Here, it may be desirable to include cell-seeded scaffolds or measurement standards to promote consistency. The reagents and scaffolds are expected to be in a form which would promote storage.

### BRIEF DESCRIPTION OF DRAWINGS

FIG. 1 shows the interconnected pore structures of lyophilized hydrogel scaffold of the present invention under SEM.

FIG. 2 shows MTT-stained MC3T3 cells evenly distributed and grown throughout the scaffold of the present invention upon seeding.

FIG. 3 shows cell adhesion and cell growth in the fibronectin-modified scaffold of the present invention, while negative controls, the non-modified scaffold and the albumen-modified scaffold do not support cell adhesion and cell growth.

FIG. 4 shows cell adhesion and cell growth in the ECM molecule-modified scaffolds of the present invention, while a negative control, the non-modified scaffold does not support cell adhesion and cell growth.

### DETAILED DESCRIPTION OF THE INVENTION

The present invention provides a method for making scaffold for cell culture having a high density of interconnected pores and being non-covalently modified with biologically active molecules. These interconnected pore structures guide and support cell and tissue growth. The pore structures provide physical surfaces, onto which the cells can lay their own ECM three-dimensionally. Moreover, the porous structures offer improved nutrient transport to the center of the scaffold through the porous interconnecting channel network and limit the cell cluster size to prevent the formation of large cell clusters that can potentially develop into necrotic center due to lack of nutrition.

Preferably, the three-dimensional scaffold used in connection with the present invention has a pore size of about 50 to 700 μm in diameter, preferably, about 75 to 300 μm in diameter. The percentage of porosity in the scaffold suitable for the non-covalent modification of the biologically active molecules is about 50% to 98%, and preferably, 80% to 95%.

The scaffold is non-covalently modified with biologically active molecules to provide interactions required for cell growth. On the scaffold, the biologically active molecules are entrapped within the porous structures, but not attached to the polymeric scaffold through covalent bonds. The biologically active molecules include ECM molecules, functional peptides, proteoglycans and glycoproteins capable of signaling cells, growth factors, and molecules for optimal cell function assayed for, and combination thereof.

When the scaffold of the present invention is functionalized with ECM molecules, it provides support and guidance for cell morphology and tissue development. The native ECM is a non-covalent three-dimensional network of proteins and polysaccharides bound together with cells intermixed. The native ECM is highly hydrated, allows for diffusion, and binds to molecules such as growth factors to allow for presentation to cells. The present invention provides a biomimetic three-dimensional environment by adding the ECM molecules onto highly hydratable structures, the lyophilized polysacchride hydrogels.

Entrapped molecules should be non-toxic, biocompatible, and the scaffold must be highly porous with large and interconnected pores and mechanically stable to resist cell contraction during tissue development. When the scaffold is non-covalently modified with growth factors, it provides cell interactive signaling for cell growth and cell culture.

The scaffold is made from lyophilization of a hydrogel of a suitable polymer. The polymer is biocompatible, either biodegradable or non-biodegradable. Preferably, the scaffold is lyophilized hydrogel of crosslinked alginate or hyaluronic acid, which is amenable to cell seeding. The pore size and distribution of the scaffold can be adjusted by changing pH, concentration of the hydrogel, or amount of crosslinker, to fit for culture of different cell types or entrapment of various bioaffecting molecules.

Alginates are linear, unbranched polymers containing β-(1→4)-linked D-mannuronic acid (M) and α-(1→4)-linked L-guluronic acid (G) residues. Alginates are produced by brown seaweed. Alginates are thermally stable cold setting gelling agents in the presence of calcium ions, which gel has lower concentrates than gelatin. Such gels can be heat treated without melting, although they may eventually degrade. The alginate polysaccharide hydrogels used in the scaffold of the present invention have several favorable properties: they are easily crosslinked and processed into three-dimensional scaffolds; they have convenient functional groups on the polymer backbone for covalent modification; the material is non-adhesive to cells in native state, which allows for the engineering of specific signals to direct cell function.

Hyaluronic acid is a natural mucopolysaccharide present at varying concentrations in practically all tissues. Aqueous solution of hyaluronic acid, the salts or derivatives thereof, or of polysaccharides in general, is characterized by notable viscosity, slipperiness, and ability to reduce friction. Such a characteristic is the basis of the presence and function of polysaccharides of the same family in the bodies of humans and other animals.

These polysaccharides are covalently crosslinked with diamines or dihydrazides as crosslinking molecules, and using the standard carbodiimide chemistry to initiate the crosslinking reaction when making the hydrogel. See for example, G. Prestwich et al., Controlled Chemical modification of hyaluronic acid: synthesis, applications, and biodegradation of hydrazide derivatives, J. Controlled Release, 1998, 53, pages 93-103. The hydrogels are thoroughly washed to remove all reactants, frozen, and lyophilized to form a three-dimensional interconnected pore network which is required for tissue engineering.

The scaffolds can be either loosely supplied on the surface of a platform or attached to the surface by covalent attachment. The hydrogel-based scaffold is covalently attached to the support substrate either via a non-fouling polysaccharide coating at the platform surface, or via amino groups terminating from the substrate surface. The biomaterial suitable for the purpose of making the cell culture scaffold of the present invention is biocompatible, either biodegradable or non-biodegradable, mechanically stable, and does not allow for protein adsorption or cell adhesion in its native unmodified state.

The scaffolds of the present invention are further modified by being impregnated with a solution containing the biologically active molecules so that the polymeric hydrogel swells and becomes entangled. When the scaffold impregnated with the solution of biologically active molecules is lyophilized, the biologically active molecules and the polymer scaffold both collapse to create interconnected and interpenetrating polymer network that is complex enough to not allow for re-solubilizing of the biologically active molecules. The biologically active molecules become physically intertwined with the polymers of the scaffolds. The polymeric entanglement is the basis for controlled release of growth factors and small molecules entrapped therein, while the high molecular weight ECM molecules have polymer chains that are long enough to stably integrate with the hydrogel scaffold and sustain cell adhesion and spreading. The length of the biologically active molecule is critical for determining the form of existence on the scaffold. If the cell-adhesive molecules are not long enough to physically entangle with the hydrogel network, these molecules can not act as anchors for cell adhesion. However, these molecules would be available to act in a soluble localized manner and control-released from the scaffold.

Preferably, the scaffold is washed thoroughly by water or a suitable buffer to adjust pH and remove salts, and then frozen and lyophilized again. The modification does not require covalent bonding. The process is simple, but still adds similar, if not better, biologically active properties to the scaffold. The biologically active molecules convey to the cells cultured on the scaffold the information and are responsible for cell adhesion interactions on the cultured cells.

The biologically active molecules suitable for entrapped in the scaffold have large molecular weight and suitable spatial configuration so that they are intertwined with the scaffold polymer or simple entrapped within the porous structures of the scaffold. The biologically active molecules may also be soluble which are reversibly entrapped in the scaffold together with the large macromolecules. When contacts or interactions occur between the entrapped biomolecules and the cells cultured on the scaffold, such interaction may not be sufficient to pull the entrapped biologically active molecules out of the scaffold.

The arrayed scaffolds can be localized or spread in a continuous manner on the surface of the platform. The platform can be a polystyrene slide or a multiwell plate. The scaffolds can be loosely placed on the platform, such as in the wells of the multiwell plate, or immobilized to the platform via a derivatized surface or a surface coating on the platform. The scaffolds can be covalently attached to the surface coating. The coating is generally a non-fouling polysaccharide. The derivatized surface generally has amino groups located on the surface that can be covalently linked with the functional groups of the scaffold polymer which has not been used up for crosslinking during the making of the scaffold.

The slide-based scaffold array is particularly useful for testing soluble environment on different non-soluble conditions, such as testing one culture medium condition on combinations of several cell types, different ECMs or peptide components within the scaffolds. The multiwell plate-based microarray is suitable for testing several different drugs on the same engineered tissue expressing molecules of interest to the pharmaceutical industry, e.g., G-protein coupled receptors, cAMP, cytochrome P450 activity. These scaffolds and engineered tissue arrays may be combined and coupled with other apparatus for testing, screening, culture purposes. For example, the array of scaffolds allows for any and all combinations of biologically active macromolecules to be non-covalently added to the scaffolds for both screening of the environments to initiate the specific signaling pathways to direct a desired biological response, such as proliferation, differentiation, angiogenesis, and to mass-produce scaffolds of any one condition for in vivo or in vitro tissue engineering.

The method for making the scaffold and microarray of the present invention are described in further details in the examples. The following example is illustrative, but not limiting the scope of the present invention. Reasonable variations, such as those occur to reasonable artisan, can be made herein without departing from the scope of the present invention.

### EXAMPLE 1

### Making the Porous Scaffold of the Present Invention

Method:

1. Three grams of alginate (MVG alginate, ProNova, Norway) were slowly dissolved in 100 ml MES buffer (pH 6.0) to obtain 3% w/v alginate solution (or pH 6.5 for use of lysine)

2. Sulfo-N-hydroxysuccinimide (Sulfo-NHS) 164 mg (MW217.13, Sigma) and 100 mg Adipic Acid Dihydrate (AAD, MW 174) were added into 50 ml 3% w/v alginate solution to obtain 15% crosslinking.

3. The alginate solution 25 ml was poured into a 50 ml conical, and 365 mg 1-ethyl-3-(3-dimethyl aminopropyl) carbodiimide (EDC, MW 191.7, Pierce) was quickly added to initiate crosslinking reaction.

4. The solution was quickly poured into an inverted petri dish with the top upside down and 2 mm spacers at sides with inverted bottom. This provided parallel surfaces separated by the 2 mm gap to gel alginate with homogeneous thickness. The material was allowed to gel overnight.

5. The hydrogel formed and was punched into several 6 mm×2 mm disks by a 6 mm biopsy punch.

6. The gel disks were rinsed in deionized water for 3 hours with 5 water changes to leach salts and reactants.

7. The gel disks were placed on plastic surface and in freezer for 4 hours, and lyophilized overnight to obtain three-dimensional porous scaffolds of the present invention.

Results:

As indicated in FIG. 1, the three-dimensional scaffold was obtained with interconnected pore structures, which was useful for further modification with bioaffecting molecules in the present invention. It was possible that the porous structures were originated from ice crystals formed during freezing, and when the ice crystals were lyophilized, the space left by the ice crystals formed interconnected porous structures. The carboxy (—COOH) groups in the hydrogel that were not crosslinked during the reaction might provide potential sites for further modification of the scaffolds.

### EXAMPLE 2

### Making the Porous Scaffold of the Present Invention

Method:

1. Two percent (w/v) alginate solution and 2% (w/v) hyaluronic acid (HA) solution in 0.1 M MES buffer (pH 6.0) were added with solution of HoBt and AAD, respectively, at 110 mg AAD/50 ml alginate/HA solution.

2. EDC dissolved in 0.1 MES buffer was added to alginate solution or hyaluronic acid solution to initiate crosslinking reactions, respectively, at 195 mg EDC/10 ml alginate/HA.

4. The solution was quickly poured into a container and allowed to gel overnight.

5. Hydrogels formed in the container and was punched into several 6 mm×2 mm disks.

6. The gel disks were rinsed in water and PBS buffer to leach out salts and reactants.

7. The gel disks were frozen and lyophilized overnight.

Results:

The three-dimensional scaffold was obtained with interconnected pore structures as lyophilized hydrogels of crosslinked alginates or hyaluronic acids. The carboxy (—COOH) groups in the hydrogel that were not crosslinked during the reaction might provide potential sites for further modification of the scaffolds. The scaffolds with interconnected pores were useful for further modification with bioaffecting molecules in the present invention.

### EXAMPLE 3

### Making the Microarray of the Present Invention

Method:

1. Following Steps 1-3 in Example 1, then, instead of pouring the gelling solution into an inverted petri dish top, the gelling solution was dispensed into wells of a 50-well silicone gasket fitted onto HA-coated polystyrene slide. Alginates hydrogel not only crosslinked in a three-dimensional arrayed configuration but also crosslinked with the surface of the slide.

2. If alginates gelled before all 50 wells could be filled with the gelling solution, one might slow down the gelling process by increasing pH or adding reactants at different times.

3. The slide was frozen and lyophilized.

Results:

The three-dimensional scaffolds were arrayed and covalently attached to the slide surface which allowed for high parallel and high throughput screening and cell culture.

### EXAMPLE 4

### Making the Microarray of the Present Invention

Method:

1. Alginate (MVG alginate, ProNova, Norway) solution 2% (w/v) was obtained by slowly dissolving alginates in 0.1 M MES buffer (pH 6.5).

2. Hydroxyl benzotiazole 68.3 mg (HoBt, H-2006, Sigma) and 110 mg AAD were added into 50 ml 2% w/v alginate solution to obtain 25% crosslinking of the carboxy groups.

3. The alginate solution aliquot in 3 ml volume was poured into a 10 ml plastic tube for reaction. The top of the tube was cut off so that the pipette tip could fit to bottom.

4. EDC 58 mg (MW 191.7, Pierce) was added into 3 ml 2% alginate solution to initiate the crosslinking reaction. The alginate solution was quickly aspirated into 0.2 ml repeat pipette tip and dispensed into wells of the 50-well gaskets placed onto 0.5% or 1.0% alginate-coated slides. Repeating the dispense 2-3 times in the same well without going over the lip of the well. PH of solution was adjusted for varying crosslinking reaction rate.

5. The slides loaded with gelling alginate solution was allowed to gel for about 20-60 minutes. Gaskets might be stacked for thicker gels.

6. The slides were frozen at −70° C. freezer for several hours or overnight and lyophilized until dry.

Results:

Scaffolds arrayed completely on the slide. Increased pH slowed down the gelling kinetics enough to allow handling of the solution prior to gelling. The gaskets were removed in most cases without disrupting the gels and keeping the gels stuck to the surface of the slide. Completely arrayed three-dimensional scaffolds of the present invention were obtained.

### EXAMPLE 5

### Making the Microarray of the Present Invention

Method:

Steps of Example 4 were repeated and in addition, pH alginate solution aliquots was adjusted to 5.5, 6.0, 6.5, and 7.0 before EDC was added to initiation the crosslinking reaction, and quality and time for the gelling process were observed and recorded.

Results:

The solution using pH 7.0 obtained a good balance between gelling quality and gelling time.

### EXAMPLE 6

### Seeding Cells on the Microarray of the Present Invention

Method:

1. Ten μl cell suspension was seeded in each scaffold having a diameter of 3 mm and a thickness of 1 mm (volume was about 7 μl) of the microarray.

2. Three scaffold arrays were superglued to the bottom of a 100 mm petri dish, and left under the laminar flow hood UV source for 20-30 minutes for sterilization.

3. Trypsinized MC3T3 cells in suspension at 10×10⁶ cells/ml were seeded onto the scaffolds by a p20 pipetteman, and 10 μl cell suspension was placed onto each scaffold.

The scaffolds sucked up the cell suspension, and cells entered the scaffolds due to capillary action and were distributed throughout the pores of the scaffolds. Twenty ml 10% FBS containing medium was added to the petri dish containing the slides for cell culture.

4. After 48 hours, cells were stained by MTT and digital images were recorded. Cells might also be observed under confocal microscope and phase contrast microscope.

Results:

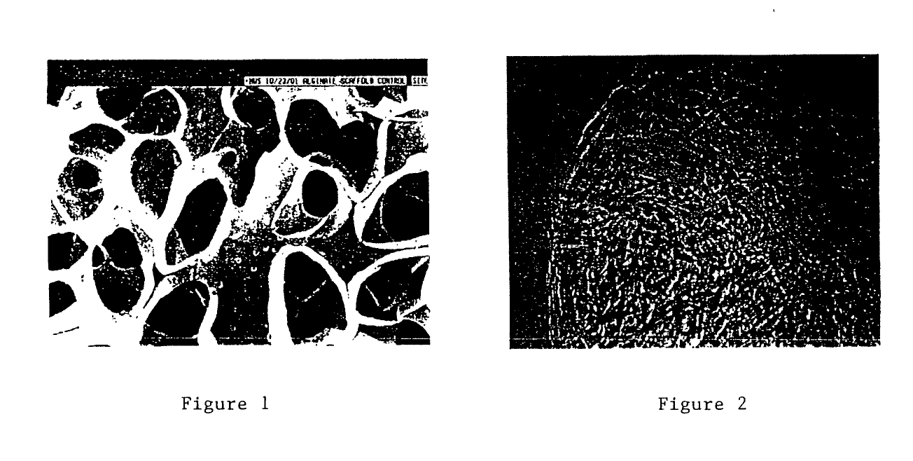

As shown in FIG. 2, cells seeded on the arrayed scaffolds of the present invention were evenly distributed throughout the scaffold and cells easily entered the open pore structures of the lyophilized scaffolds without interaction with the alginate scaffold. It also demonstrated the interconnectivity of the scaffolds.

### EXAMPLE 7

### Seeding Cells on the Arrayed Scaffolds of the Present Invention

Method:

1. Trypsinized and suspended MC3T3 cells were prepared at 0.5, 1.0, 5.0, and 10×10⁶ cells/ml.

2. Cell suspension 60 μl was seeded onto each scaffold (56.5 μl in volume) of a microarray on a 24-well plate by placing a tip of a P200 pipetteman loaded with cell suspension in the middle of the scaffold and dispensing the cell suspension into the scaffold.

3. Culture medium 0.5 ml was added to each well and cells were cultured under proper conditions.

4. Twenty hours later, cells were stained with MTT 10% (v/v) for observation.

Results:

Seeded cells were distributed throughout the alginate scaffold along the entire thickness, and the cells existed mainly as clumps of cells. As the focal plane was changed on the microscope, new cell aggregates appeared in focus. The adhesion of the cells to each other was most likely due to cells not being able to adhere to the alginate scaffold. Incorporating cell adhesion molecules, full ECM proteins, or peptide ligands, might cause dramatic morphological changes in the cells that could be easily assayed for on these scaffolds.

Initial cell concentration and porous structure of the scaffolds had effects on cell seeding distribution. The smaller the pore, the more the cell aggregates with fewer cells than the aggregates in the larger-pored scaffolds. The larger pored scaffolds had larger clumps of cells and fewer in number. It demonstrated that the three-dimensional scaffolds of the present invention were useful for cell seeding and three-dimensional cell growth and cell culture.

### EXAMPLE 8

### Cell Culture on Modified Scaffold of the Present Invention

Method:

1. Scaffolds of lyophilized of hydrogels of crosslinked alginate and hyaluronic acid were impregnated with 0.1 mg/ml collagen I solution in acid buffer.

2. Impregnated scaffolds were either unwashed or washed in PBS and water for 4 hours.

3. Washed or unwashed scaffolds were frozen at −70° C. for several hours and lyophilized.

4. Trypsinized MC3T3 cells 50 μl at 4×10⁶ cells/ml were seeded onto each scaffold by P200 Pipetteman to obtain a cell density of approximately 200,000 cells per scaffold. The cell suspension was filled in the pipette tip, and when the end of the tip penetrated the scaffold, the cell suspension was simultaneously injected into the scaffold.

5. The scaffolds seeded with cells were transferred into a plate with 200 μl culture medium (aMEM+10% FBS) and maintained at 37° C. in incubator and observed continuously.

6. Cells might be trypsinized and collected for count for cell growth. Alternatively, cells grown on the scaffolds were observed under the microscope and sampled every day for examination on cell morphology and cell growth. The scaffolds with cells grown thereon were stained by conventional method for cell viability such as MTT. Cell suspension without any scaffolds was observed under the same conditions as control. Kit L-3224 by Molecular Probes was also used to assay for cell viability.

Results:

Cell attachment and cell growth were observed on the alginate or hyaluronic acid scaffold modified with collagen of the present invention, while scaffold with no collagen and the cell suspension did not support cell attachment and cell growth. Cells attached and spread on the modified scaffold pores which was necessary for cell proliferation, while cells in non-modified scaffolds existed as multicellular aggregates because they could not adhere to the scaffolds.

Scaffolds with non-covalently modified ECM molecules of the present invention supports cell adhesion and cell growth, while in their non-modified states, these scaffolds did not support cell adhesion and cell growth. The non covalent modification method of the present invention thus promoted the function of the cell culture scaffolds for cell attachment and cell growth.

### EXAMPLE 9

### Cell Culture on the Modified Scaffold of the Present Invention

Method:

1. Three-dimensional alginate scaffolds of the present invention were modified with fibronectin (Human fibronectin in PBS, from Becton Dickinson Labware) or Bovine serum albumen (BSA, fraction V, Sigma IIA-7906). The concentrations of fibronectin and BSA solutions for impregnation of the scaffolds and non-covalent modification were both 100 μg/ml. After being impregnated with the solutions, the scaffolds were frozen and lyophilized.

2. The scaffolds were seeded with MC3T3 cells at 100,000 cells/scaffold.

3. The scaffolds seeded with cells were cultured at proper conditions and observed continuously and stained by MTT at the end for cell viability.

Results:

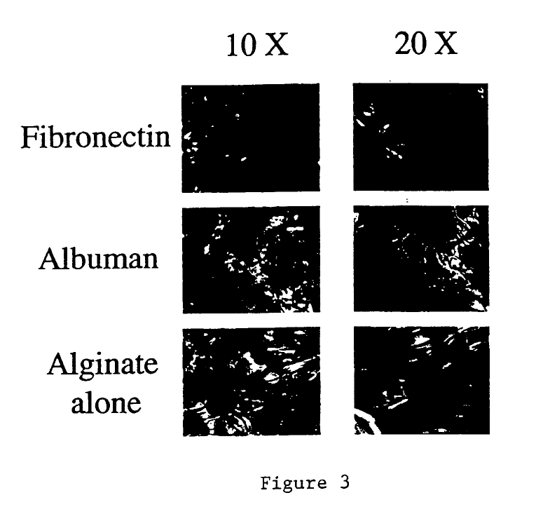

As shown in FIG. 3, cell attachment was observed on fibronectin-modified scaffolds of the present invention, while the scaffolds modified with albumen or non-modified scaffolds did not support such cell attachment or promote cell adhesion. Cell growth was observed on fibronectin-modified scaffolds of the present invention, while the scaffolds modified with albumen or non-modified scaffolds did not have cell growth.

Fibronectin belonged to the ECM proteins known to promote cell adhesion and cell attachment, while BSA, a large protein similar to fibronectin in size, did not support cell adhesion and cell attachment. It was the scaffolds modified with fibronectin, not BSA, that promoted cell adhesion and cell growth. The scaffolds modified with BSA and the non-modified scaffolds, as negative controls, further confirmed that the ECM molecule-modified scaffolds of the present invention had function of promoting cell adhesion and cell growth.

### EXAMPLE 10

### Cell Culture in the Arrayed Programmable Scaffold of the Present Invention

Method:

1. Polystyrene slides were coated with polyethyleneimine (PEI) and hyaluronic acid (HA) using polystyrene housing as a mask.

2. Masks from Grace Biolab were used to array EDC/AAD crosslinked HA scaffolds.

3. Three-dimensional HA scaffold arrays with interconnected pore structures were obtained by lyophilization as described above.

4. The lyophilized scaffold arrays were hydrated with solutions containing ECM molecules including human fibronectin (100 μg/ml, BD Labware), mouse laminin (100 μg/ml, BD Labware), Collagen IV (100 μg/ml, BD Labware), respectively. Then, the hydrated scaffold arrays were frozen and lyophilized to obtain modified scaffold arrays.

5. MC3T3 cells were seeded at 2×10⁶ cells/ml, 2-3 μl per arrayed scaffold. The slide reservoir was filled with 5 ml culture medium and cultured for 3-4 days.

6. Cells were stained with MTT for viability.

7. The cells were also stained with propidium iodide for fluorescent staining of the nuclei, and observed under the Universal Imaging System for photograph.

Results:

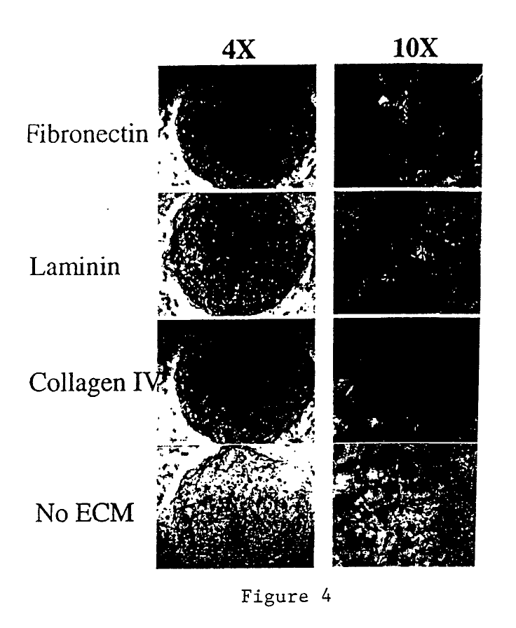

As shown in FIG. 4, cells formed attachment to the scaffolds of the microarray of the present invention modified with ECM molecules, and there was no cell attachment or cell growth observed on non-modified scaffolds. ECM molecule-modified scaffolds of the present invention supported cell adhesion and cell growth, and these modified scaffolds, when in an array, were useful for assays and screening for microenvironment for cell signaling and cell growth.

### EXAMPLE 11

### Cell Culture in the Modified Arrayed Scaffolds of the Present Invention

Method:

1. Arrayed alginate scaffolds of the present invention were modified with human fibronectin at 100 μg/ml, or mouse laminin (Gibep) at 100 μg/ml, or Matrigel (Becton Dickinson) at 50 μg/ml. ECM or Matrigel solution 1 μl was used to impregnate each scaffold.

Matrigel is a trademark for a solubilized basement membrane preparation extracted from the Engelbreth-Holm-Swarm (EHS) mouse sarcoma, a tumor rich in extracellular matrix proteins. The product is commercially available from Becton Dickinson Bioscience. Its major component is laminin, followed by collagen IV, entactin, and heparan sulfate proteoglycan. It also contains TGF-β fibroblast growth factor, tissue plasminogen activator, and other growth factors which occur naturally in the EHS tumor. At room temperature, Matrigel Matrix polymerizes to produce biologically active matrix material resembling the mammalian cellular basement membrane. Matrigel Basement Membrane Matrix is effective for the attachment and differentiation of both normal and transformed anchorage dependent epithelial and other cell types. These include neurons, hepatocytes, Sertoli cells, mammary epithelial, melanoma cells, vascular endothelial cells, thyroid cells and hair follicle cells.

2. The scaffolds were seeded with HEPG2 cells or MC3T3 cells at 100,000 cells per scaffold and cultured in 10% serum-containing medium for 1 week.

3. The scaffolds were maintained and observed continuously. Cells were stained by MTT for cell viability and also recorded by phase contrast microscopy.

Results:

ECM or Matrigel modified scaffolds of the present invention supported cell adhesion and cell growth of cells from different tissue (hepatocytes and osteoblasts) and different species (mouse and human). The array of the modified scaffolds allowed the high parallel and high throughput screening for such microenvironment for cell culture on different cell types as well as differed cell culture soluble environment.

### EXAMPLE 12

### Cell Culture in the Modified Arrayed Scaffolds of the Present Invention

Method:

1. As in Example 11, arrayed alginate scaffolds of the present invention were modified with human fibronectin at 100, 30, 10, 3, and 1 μg/ml in PBS, or mouse laminin (Gibco) at 100, 30, 10, 3, and 1 μg/ml in PBS, or mouse collagen IV at 100, 30, 10, 3, and 1 μg/ml. ECM solution 1 μl was used to impregnate each scaffold.

2. The scaffolds were seeded with cells at 100,000 cells per scaffold, cultured, and observed continuously.

Results:

ECM-modified scaffolds of the present invention supported cell adhesion and cell growth of cells at various concentrations. The array of the modified scaffolds allowed the high parallel and high throughput screening for such microenvironment for cell culture on different cell types as well as differed cell culture soluble environment.

We claim:

1. A method for programming a scaffold for cell culture comprising
impregnating a porous scaffold with a solution containing biologically active molecules, and lyophilizing the impregnated scaffold so that the biologically active molecules are entrapped within the porous scaffold.

2. The method of claim 1, further comprising washing the impregnated scaffold to remove salt and adjust pH before lyophilization.

3. The method of claim 1, wherein the porous scaffold is a three-dimensional scaffold having interconnected pore structures.

4. The method of claim 3, wherein the porous scaffold has pore size of about 50 to 700 μm in diameter.

5. The method of claim 4, wherein the porous scaffold has pore size of about 75 to 300 μm in diameter.

6. The method of claim 3, wherein the porous scaffold has a porosity of about 50% to 98%.

7. The method of claim 6, wherein the porous scaffold has a porosity of about 80% to 95%.

8. The method of claim 1, wherein the porous scaffold is a polymeric, ceramic, metallic, or composite material.

9. The method of claim 1, wherein the porous scaffold is biocompatible.

10. The method of claim 1, wherein the porous scaffold is biodegradable.

11. The method of claim 1, wherein the porous scaffold is a lyophilized polymeric hydrogel.

12. The method of claim 11, wherein the polymer is a crosslinked alginate or crosslinked hyaluronic acid.

13. The method of claim 1, wherein the porous scaffold is made from a polymer selected from alginate, hyaluronic acid, agarose, collagen, chitosan, chitin, polytrimethylene carbonate, poly hydroxybutyrate, amino acid-based polycarbonates, poly vinylchloride, polyHEMA, polystyrene, PTFE, poly ethylene glycol, or polypropylene glycol-based polymers.

14. The method of claim 1, wherein the porous scaffold is made from a biodegradable polymer selected from poly lactides, glycolides, caprolactones, orthoesters, or copolymers thereof.

15. The method of claim 1, wherein the biologically active molecules are entrapped with in the porous scaffold.

16. The method of claim 1, wherein the biologically active molecules include extracellular matrix (ECM) molecules, functional peptides, proteoglycans and glycoproteins capable of signaling cells, growth factors, molecules for optimal cell function, and combinations thereof.

17. The method of claim 16, wherein the ECM molecules are selected from fibronectin, laminin, collagen, thrombospondin 1, vitronectin, elastin, tenascin, aggrecan, agrin, bone sialoprotein, cartilage matrix protein, fibronogen, fibrin, fibulin, mucins, entactin, osteopontin, plasminogen, restrictin, serglycin, SPARC/osteonectin, versican, von Willebrand Factor, polysacchride heparin sulfate, cell adhesion molecules including cadherins, connexins, selectins, or combinations thereof.

18. The method of claim 16, wherein the growth factor is selected from epidermal growth factor, fibroblast growth factor, platelet-derived growth factor, nerve growth factor, transforming growth factor-β, hematopoictic growth factors, interleukins, or combinations thereof.

19. A programmable scaffold made by the method as described in claim 1.

20. A method for making an array of scaffolds comprising
arranging a series of programmable scaffolds according to claim 19 on a platform to from the array; or
distributing a solution of polymer(s) on a platform to form a series of solution spots,
crosslinking the polymer to form crosslinked hydrogel, and lyophilizing to form the array of scaffolds.

21. The method of claim 20, wherein crosslinking is adjusted by changing pH.

22. The method of claim 20, wherein the polymer solution comprises a diamine at an amount of about 2% to 100% molar ratio of functional groups to hyaluronic acid or alginate.

23. The method of claim 22, wherein the diamine is at an amount of about 10% to 40% molar ratio of functional groups to hyaluronic acid or alginate.

24. The method of claim 23, wherein the diamine is lysine or adipic dihydrazide.

25. The method of claim 20, wherein the polymeric solution comprises a carbodiimide at an amount of about 25% to 200% molar ratio of functional groups to hyaluronic acid or alginate.

26. The method of claim 25, wherein the carbodiimide is at an amount of about 50% to 100% molar ratio of functional groups to hyaluronic acid or alginate.

27. The method of claim 26, further comprising a coreactant selected from the group consisting of HoBt, NHS, and sulfo NHS.

28. The method of claim 27, wherein the coreactant is at a ratio of about 1:50 to 50:1 to the carbodiimide.

29. The method of claim 28, wherein the coreactant is at a ratio of about 1:10 to 4:1 to the carbodiimide.

30 The method of claim 20, further comprising
impregnating the array of scaffolds with a solution containing biologically active molecules, and lyophilizing the impregnated array of scaffolds so that the biologically active molecules are entrapped within the scaffolds.

31. An array of scaffolds made by the method as described in claim 20.

32. The array of claim 31, wherein the platform is a slide or a multi-well plate.

33. The array of claim 31, wherein the scaffolds are loosely placed on the platform.

34. The array of claim 31, wherein the scaffolds are attached to platform through covalent attachment.

35. The array of claim 34, wherein the platform is coated with a substrate surface, and the multiplicity of scaffolds are attached to the platform through covalent bonding to the substrate surface.

36. The array of claim 35, wherein the substrate is a non-fouling polysaccharide.

37. The array of claim 36, wherein the substrate has a terminal amino group to which the multiplicity of scaffolds attach.

38. A kit comprising the array of claim 31.

39. The kit of claim 38, further comprising seeded cells on scaffolds.

40. The kit of claim 38, wherein the pore structures contain molecules to be assayed for.

41. In a method for cell culture wherein the improvement comprises culturing cells on the scaffolds of claim 19.

42. In a screening method wherein the improvement comprises screening on the scaffold of claim 19.

43. The method of claim 42, wherein the screening molecule is a cell-interactive signaling biomolecule or a pharmaceutical compound.

44. The method of claim 42, wherein the screening molecule is a soluble molecule in cell culture medium.

45. The method of claim 42, wherein the screening is for cells of different types grown on the multiplicity of the scaffolds.
